(19) United States
(12) Patent Application Publication
(10) Pub. No.: US 2024/0076454 A1
(43) Pub. Date: Mar. 7, 2024

(54) BIOERODIBLE CROSSLINKING HYDROGEL BASED ON MULTI-ARM POLYOXAZOLINES WITH CAGE-LIKE SILICON-OXYGEN CORES

(71) Applicant: Boston Scientific Scimed, Inc., Maple Grove, MN (US)

(72) Inventors: Joseph Thomas Delaney, JR., Minneapolis, MN (US); Yen-Hao Hsu, Shrewsbury, MA (US)

(73) Assignee: Boston Scientific Scimed, Inc., Maple Grove, MN (US)

(21) Appl. No.: 18/450,122

(22) Filed: Aug. 15, 2023

Related U.S. Application Data
(60) Provisional application No. 63/399,122, filed on Aug. 18, 2022.

(51) Int. Cl. C08G 83/00; A61L 27/18; A61L 27/52; A61L 27/58

(52) U.S. Cl. CPC C08G 83/006; A61L 27/18; A61L 27/52; A61L 27/58; C08G 83/005; A61L 2400/06

(57) ABSTRACT

In some aspects, the present disclosure pertains to reactive multi-arm polymers having a cage-like silicon-oxygen core and a plurality of polyoxazoline-containing arms extending from the core, in which the polyoxazoline-containing arms comprise a first end that is covalently attached to the cage-like silicon-oxygen core and a second end comprising a moiety that comprises a reactive end group. In other aspects, the present disclosure pertains to systems that comprise such reactive multi-arm polymers and multifunctional compounds that comprise functional groups that are reactive with the reactive end groups of the reactive multi-arm polymers. Other aspects pertain to medical hydrogels formed by crosslinking such reactive multi-arm polymers with such multifunctional compounds and methods of treatment that comprise administering to a subject a mixture that comprises and such reactive multi-arm polymers with such multifunctional compounds.

### CROSS-REFERENCE TO RELATED APPLICATIONS

This application claims the benefit of U.S. Provisional Patent Application Ser. No. 63/399,122 filed on Aug. 18, 2022, the disclosure of which is incorporated herein by reference.

### FIELD

The present disclosure relates to multi-armed polyoxazolines having cage-like silicon-oxygen cores, bioerodible crosslinked compositions containing such multi-armed polymers, methods of making such multi-armed polymers, and methods of using such multi-armed polymers, among other aspects. The multi-armed polyoxazolines having cage-like silicon-oxygen cores of the present disclosure are useful, for example, in various biomedical applications.

### BACKGROUND

Bioerodible injectable hydrogels are a newly emerging class of materials having a variety of medical uses.

As one specific example, in the case of SpaceOAR®, a long-term bioerodible injectable hydrogel based on multi-arm PEG-based, such products are used to create or maintain space between tissues in order to reduce side effects of off-target radiation therapy. See “Augmenix Announces Positive Three-year SpaceOAR Clinical Trial Results,” Imaging Technology News, Oct. 27, 2016. As another specific example, Augmenix has developed TraceIT® Hydrogel, a bioerodible injectable hydrogel synthetic hydrogel consisting primarily of water and iodinated crosslinked polyethylene glycol (PEG) that is visible under CT, cone beam, ultrasound and MR imaging and is useful as a tissue marker (e.g., for targeted radiation therapy). See “Augmenix Receives FDA Clearance to Market its TraceIT™ Tissue Marker,” BusinessWire Jan. 28, 2013. TraceIT® hydrogel remains stable and visible in tissue for three months, long enough for radiotherapy, after which it is absorbed and cleared from the body. Id.

There is a continuing need in the biomedical arts for additional hydrogels, including bioerodible injectable hydrogels, for precursors of such hydrogels, for methods of making such hydrogels and precursors, for methods of using such hydrogels and precursors, and for systems for forming such hydrogels, among other needs.

### SUMMARY

In some aspects, the present disclosure pertains to reactive multi-arm polymers having a cage-like silicon-oxygen core and a plurality of polyoxazoline-containing arms extending from the core, in which the polyoxazoline-containing arms comprise a first end that is covalently attached to the cage-like silicon-oxygen core and a second end comprising a moiety that comprises a reactive end group.

In some embodiments, the cage-like silicon-oxygen core is selected from a T₆ cage-like silicon-oxygen core, a T₈ cage-like silicon-oxygen core, a T₁₀ cage-like silicon-oxygen core and a T₁₂ cage-like silicon-oxygen core and/or the polyoxazoline-containing arms comprise polymerized monomers selected from oxazoline, 2-(C₁-C₁₀-alkyl)-2-oxazolines, and combinations thereof.

In some embodiments, which can be used in conjunction with any of the above aspects and embodiments, the reactive end groups are electrophilic groups. Examples of electrophilic groups may be selected, for example, from N-hydroxysuccinimidyl esters, imidazole esters, imidazole carboxylates and benzotriazole esters, among others.

In some embodiments, which can be used in conjunction with any of the above aspects and embodiments, the moiety that comprises the reactive end group may further comprise a hydrolysable ester group.

In some embodiments, which can be used in conjunction with any of the above aspects and embodiments, the moiety that comprises the reactive end group comprises a diester selected from a malonic-acid-based diester, a succinic-acid-based diester, a glutaric-acid-based diester and an adipic-acid-based diester.

I some aspects, the present disclosure pertains to systems that comprise (a) a reactive multi-arm polymer in accordance with any of the above aspects and embodiments, and (b) a multifunctional compound that comprises functional groups that are reactive with the reactive end groups of the reactive multi-arm polymer.

In some embodiments, which can be used in conjunction with any the above aspects, the reactive groups of the reactive multi-arm polymer are electrophilic groups and the functional groups of the multifunctional compound are nucleophilic groups.

In some embodiments, which can be used in conjunction with any of the above aspects and embodiments, multifunctional compound may be a polyamine compound.

In some embodiments, which can be used in conjunction with any of the above aspects and embodiments, the multifunctional compound may be a polyamine compound that comprises residues of from 2 to 10 basic amino acids.

In some embodiments, which can be used in conjunction with any of the above aspects and embodiments, the multifunctional compound may be a polyamine compound that comprises a plurality of —(CH₂)ₓ—NH₂ groups where x is 0, 1, 2, 3, 4, 5 or 6.

In some embodiments, which can be used in conjunction with any of the above aspects and embodiments, the multifunctional compound may be a polyamine compound that comprises two or more amino acid residues selected from residues of lysine, ornithine, and combinations thereof.

In some embodiments, which can be used in conjunction with any of the above aspects and embodiments, the system may comprise a first precursor composition that comprises the multifunctional compound and a second precursor composition that comprises the reactive multi-arm polymer.

In some embodiments, which can be used in conjunction with any of the above aspects and embodiments, the system may further comprise an accelerant composition. For example, the accelerant composition may comprise a buffer solution having a pH ranging from about 9 to about 11.

In some embodiments, which can be used in conjunction with any of the above aspects and embodiments, the first precursor composition may be provided in a syringe barrel, the second precursor composition may be provided in a vial, and the accelerant composition may be provided in a syringe barrel.

In some embodiments, which can be used in conjunction with any of the above aspects and embodiments, the system may further comprise a delivery device.

In some aspects, the present disclosure pertains to medical hydrogels formed by crosslinking a reactive multi-arm polymer, in accordance any of the above aspects and embodiments, with a multifunctional compound that comprises functional groups that are reactive with the reactive end groups of the reactive multi-arm polymer, in accordance any of the above aspects and embodiments.

In some aspects, the present disclosure pertains to methods of treatment comprising administering to a subject a mixture that comprises a reactive multi-arm polymer, in accordance any of the above aspects and embodiments, a multifunctional compound that comprises functional groups that are reactive with the reactive end groups of the reactive multi-arm polymer, in accordance any of the above aspects and embodiments.

In addition to the above, further aspects and embodiments of the present disclosure will become readily apparent upon review of the Detailed Description to follow.

### BRIEF DESCRIPTION OF THE DRAWINGS

FIG. 1A schematically illustrates a method in which a silsesquioxane hydride is used to form a hydroxyalkyl silsesquioxane, in accordance with an embodiment of the present disclosure.

FIG. 1B schematically illustrates a method in which the hydroxyalkyl silsesquioxane FIG. 1A is used to form a haloalkyl silsesquioxane, in accordance with an embodiment of the present disclosure.

FIG. 1C schematically illustrates a method in which the haloalkyl silsesquioxane of FIG. 1B is used as an initiator for a ring-opening polymerization to form multi-arm polyoxazoline having terminal hydroxyl groups, in accordance with an embodiment of the present disclosure.

FIG. 1D schematically illustrates a method in which the multi-arm polyoxazoline of FIG. 1C is subsequently converted into a multi-arm polyoxazoline that is terminated with reactive succinimidyl glutarate groups, in accordance with an embodiment of the present disclosure.

FIG. 2 is a schematic illustration of a method of making a reactive multi-arm polyoxazoline, in accordance with an embodiment of the present disclosure.

FIG. 3 is a schematic illustration of a method of crosslinking a reactive multi-arm polyoxazoline with a multifunctional crosslinking agent, in accordance with an embodiment of the present disclosure.

### DETAILED DESCRIPTION

In some aspects, the present disclosure pertains to multi-arm polymers having a cage-like silicon-oxygen core and having a plurality of polyoxazoline-containing arms.

In various embodiments the multi-arm polymers are silsesquioxane compounds having a plurality of polyoxazoline-containing arms. A silsesquioxane is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polyoxazoline-containing arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices. The silicon-oxygen cores are sometimes referred to as T₆, T₈, T₁₀, and T₁₂ cage-like silicon-oxygen cores, respectively (where T=the number of tetrahedral Si vertices). In all cases each Si atom is bonded to three O atoms, which in turn connect to other Si atoms.

Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric groups, among others. The T₈ cage-like silicon-oxygen cores are widely studied and have the formula [RSiO_{3/2}]₈, or equivalently R₈Si₈O₁₂. Such a structure is shown here:

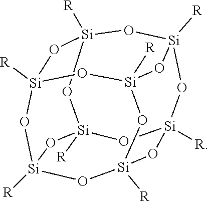

In the present disclosure, at least one R group is a polyoxazoline-containing arm, and typically all R groups are polyoxazoline-containing arms.

In various embodiments, the polyoxazoline-containing arms (e.g., the R groups in the above silsesquioxane formulas and structure, among many other possibilities) comprise one or more polymerized monomers selected from oxazoline and 2-alkyl-2-oxazolines, for instance, 2-(C₁-C₁₀alkyl)-2-oxazolines, including 2-methyl-2-oxazoline, 2-ethyl-2-oxazoline, 2-propyl-2-oxazoline, 2-butyl-2-oxazoline (including 2-n-butyl-2-oxazoline and 2-sec-butyl-2-oxazoline isomers), 2-pentyl-2-oxazoline (including various isomers), 2-hexyl-2-oxazoline (including various isomers), and so forth.

In various embodiments, the polyoxazoline-containing arms comprise a first end that is covalently attached to the cage-like silicon-oxygen core (e.g., a T₆, T₈, T₁₀ or T₁₂ cage-like silicon-oxygen core) and a second end comprising a moiety that comprises a reactive end group. (Such molecules are also referred to herein as reactive multi-arm polyoxazolines.) In some of these embodiments, the reactive end groups may be electrophiles, for example, selected from N-hydroxysuccinimide esters, imidazole esters, imidazole carboxylates and benzotriazole esters, among other possibilities. In some of these embodiments, the reactive end groups may be nucleophiles, for example, selected from amine groups or thiol groups, among other possibilities.

In some embodiments, the moiety that comprises the reactive end group may further comprise a hydrolysable ester group. For instance, the moiety that comprises a reactive end group may comprise a diester. In particular examples, the diester may be selected from a malonic-acid-based diester, a succinic-acid-based diester, a glutaric-acid-based diester and an adipic-acid-based diester.

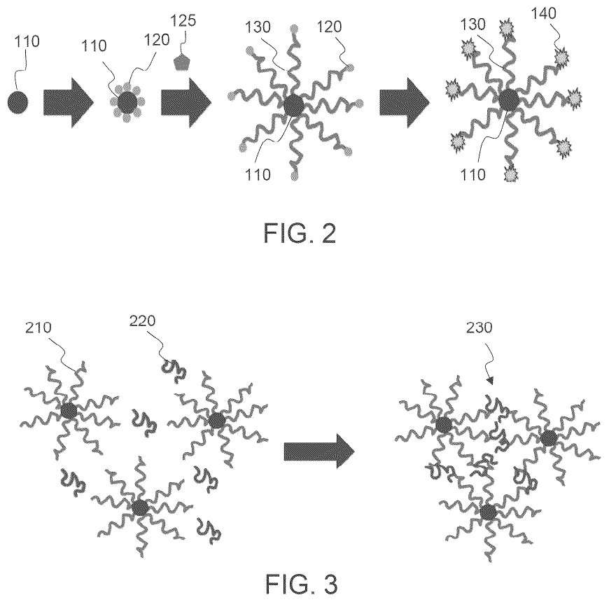

The formation the above and other multi-arm polyoxazolines is shown schematically in FIG. 2, in which a molecule with a cage-like silicon-oxygen core 110 (e.g., a silsesquioxane core) is used to form a multifunctional initiator molecule 120, specifically, a molecule with a cage-like silicon-oxygen core 110 and multiple moieties that comprises a polymerization initiator group 120 from which oxazoline polymerization can proceed. Then, polymerization of an oxazoline monomer 125 proceeds from the multifunctional initiator molecule 120 to form a multi-arm polyoxazoline that comprises a cage-like silicon-oxygen core 110 and a plurality of polyoxazoline-containing arms 130 extending therefrom, each having a first end and a second end, wherein the first end is linked to the core 110. Subsequently, a reactive group 140 may be provided at the second end of each polyoxazoline-containing arm 130. In certain embodiments, reactive group 140 is linked to the polyoxazoline-containing arm 130 by a hydrolysable ester group.

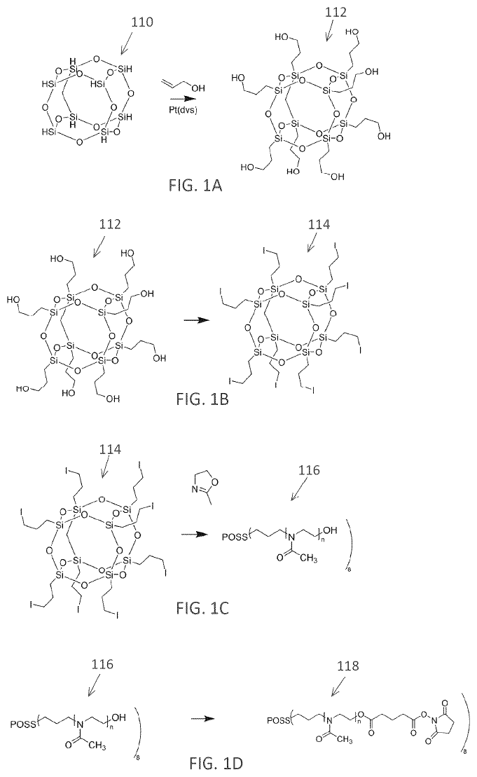

In particular embodiments, a silsesquioxane precursor molecule that has a cage-like silicon-oxygen core (e.g., a T₆, T₈, T₁₀ or T₁₂ cage-like silicon-oxygen core, among other possibilities), for example, a silsesquioxane hydride of the chemical formula [RSiO_{3/2}]ₙ, where n is an integer of at least 6 and where R═H, is reacted with a vinyl substituted C₃-C₁₀ alcohol such as 2-propene-1-ol (also known as allyl alcohol), 3-butene-1-ol, 4-pentene-1-ol, or 5-hexene-1-ol, and so forth, to form a C₃-C₁₀ hydroxyalkyl-substituted silsesquioxane, for instance, an omega-hydroxyalkyl-substituted silsesquioxane, such as a compound of the chemical formula [RSiO_{3/2}]ₙ, where n is an integer of at least 6, R is —(CH₂)ₘOH, and m ranges from 3 to 10, e.g., a 3-hydroxypropyl-substituted silsesquioxane (e.g., where R=—CH₂CH₂CH₂OH), a 4-hydroxybutyl-substituted silsesquioxane (e.g., where R=—CH₂CH₂CH₂CH₂OH), a 5-hydroxypentyl-substituted silsesquioxane (e.g., where R=—CH₂CH₂CH₂CH₂CH₂OH), a 6-hydroxyhexyl-substituted silsesquioxane (e.g., where R=—CH₂CH₂CH₂CH₂CH₂CH₂OH), and so forth. A particular example such a reaction step is shown in FIG. 1A, which schematically illustrates a method in which a T₈ silsesquioxane hydride of the formula [RSiO_{3/2}]₈ where R is H is used to form a hydroxyalkyl T₈ silsesquioxane of the formula [RSiO_{3/2}]₈, where R is —CH₂CH₂CH₂OH.

Then, the hydroxyl groups of the C₃-C₁₀ hydroxyalkyl-substituted silsesquioxane are converted to halogen groups by reaction with a halogen (e.g., Cl₂, Br₂, I₂) to form a C₃-C₁₀ haloalkyl-substituted silsesquioxane, for instance, an omega-haloalkyl-substituted silsesquioxane such as a compound of the chemical formula [RSiO_{3/2}]ₙ, where n is an integer of at least 6, R is —(CH₂)ₘX, m ranges from 3 to 10, and X═Cl, Br, or I, e.g., a 3-halopropyl-substituted silsesquioxane (e.g., where R=—CH₂CH₂CH₂X), a 4-halobutyl-substituted silsesquioxane (e.g., where R=—CH₂CH₂CH₂CH₂X), a 5-halopentyl-substituted silsesquioxane (e.g., where R=—CH₂CH₂CH₂CH₂CH₂X), a 6-halohexyl-substituted silsesquioxane (e.g., where R=—CH₂CH₂CH₂CH₂CH₂CH₂X), and so forth. A particular example such a reaction step is shown in FIG. 1B, which schematically illustrates a method in which the hydroxyalkyl silsesquioxane FIG. 1A is used to form a haloalkyl T₈ silsesquioxane of the formula [RSiO_{3/2}]₈, where R is —CH₂CH₂CH₂I.

The C₃-C₁₀ haloalkyl-substituted silsesquioxane is then used as an initiator for the ring-opening polymerization an oxazoline monomer, for example, oxazoline or a 2-alkyl-2-oxazoline as described above, followed by termination/quenching of the ring-opening polymerization, such that a polyoxazoline arm is formed at each halide atom, resulting in a silsesquioxane having a plurality of hydroxyl-terminated polyoxyazoline arms, for example, a polyoxazoline that comprises a cage-like silicon-oxygen core (e.g., cage-like silicon-oxygen core such as a T₆, T₈, T₁₀ or T₁₂ core, etc.) and a plurality of polyoxazoline-containing arms, each having a first end and a second end, formed from at least one type of oxazoline monomer, wherein the first end is linked to the core and the second end comprises a hydroxyl group. A particular example such a reaction step is shown in FIG. 1C, which schematically illustrates a method in which the haloalkyl silsesquioxane of FIG. 1B is used as an initiator to for a ring-opening polymerization reaction of 2-methyl-2-oxazoline through which is formed a multi-arm polyoxazoline having a core in the form of a T₈ cage-like silicon-oxygen core (represented by POSS in FIG. 1C) of the formula [RSiO_{3/2}]₈, where R is

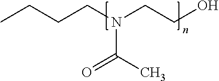

and n ranges from 10 to 25. It is noted that while halogen-based groups are exemplified in the above reaction steps, other leaving groups including methylsulfonate/mesylate and tosylate groups may be used as well.

The terminal hydroxyl groups of the polyoxyazoline arms of the resulting silsesquioxane are available for subsequent reactions, for example, reactions that provide can reactive end groups such as reactive electrophilic groups (e.g., N-hydroxysuccinimide esters groups, such as succinimidyl glutarate groups, succinimidyl succinate groups, succinimidyl carbonate groups, or succinimidyl adipate groups, imidazole ester groups, imidazole carboxylate groups and/or benzotriazole ester groups, among others) or reactive nucleophilic groups (e.g. amine groups and/or thiol groups, among others).

In particular embodiments, terminal hydroxyl groups of the polyoxyazoline arms of the resulting silsesquioxane are reacted with a cyclic anhydride (e.g., glutaric anhydride, succinic anhydride, malonic anhydride, etc.) to form a reaction product in the form of a silsesquioxane, which has a cage-like silicon-oxygen core that comprises a plurality of polyoxazoline-containing arms having a first end linked to the core and a second end comprises moiety that comprises a carboxylic acid group that is connected to the polyoxazoline-containing arm though a hydrolysable ester group, which is then treated with a coupling agent (e.g., a carbodiimide coupling agent such as N,N′-dicyclohexylcarbodiimide (DCC), 1-Ethyl-3-(3-dimethylaminopropyl)carbodiimide (EDC), N-Hydroxybenzotriazole (HOBt), BOP reagent, and/or another coupling agent) and N-hydroxysuccinimide (NHS), to yield a reactive multi-arm polyoxazoline comprising succinimidyl end groups, in particular, a hydrolysable ester group and a reactive succinimidyl ester end group, such as a succinimidyl glutarate group, succinimidyl succinate group, succinimidyl carbonate group, or succinimidyl adipate group. A particular example such a reaction step is shown in FIG. 1D, which schematically illustrates a method in which the hydroxyl groups of the multi-arm polyoxazoline of FIG. 1C are reacted with glutaric anhydride, followed by carbodiimide coupling with N-hydroxysuccinimide (NHS), to produce a multi-arm polyoxazoline that is terminated with reactive succinimidyl glutarate groups.

Using the above and other techniques, reactive multi-arm polyoxazolines may be formed, which comprise a cage-like silicon-oxygen core (e.g., cage-like silicon-oxygen core such as a T₆, T₈, T₁₀ or T₁₂ core, etc.) and a plurality of polyoxazoline-containing arms, each having a first end and a second end, and each formed from polymerization of at least one type of oxazoline monomer, wherein the first end is linked to the cage-like silicon-oxygen core and the second end comprises a reactive group.

In some embodiments, at least a portion of the polyoxazoline arms may comprise one or more covalently linked radiopaque moieties, for example, bromine or iodine groups. For instance, an iodine-containing moiety may be linked to at least a portion of the polyoxazoline arms by a suitable covalent linkage, such as an ester or amide linkage, among others. Examples of such iodine-containing moieties include aromatic moieties that comprise a monocyclic or multicyclic aromatic structure, such as a benzene group or a naphthalene group, that is substituted with the following: one or more radiopaque functional groups, for example, one or more iodine groups and, optionally, a plurality of hydrophilic functional groups, for example, hydrophilic functional groups selected from one or more of hydroxyl groups, C₁-C₄-hydroxyalkyl groups, C₁-C₄-aminoalkyl groups or C₁-C₄-carboxyalkyl groups.

Reactive multi-arm polyoxazolines as described herein may be crosslinked with a suitable crosslinking agent, either in vivo or ex vivo, to form a crosslinked product. The crosslinked product may be in the form of a hydrogel when hydrated.

In some embodiments, the reactive multi-arm polyoxazolines may be crosslinked with multifunctional compounds having functional groups that are reactive with the reactive groups of the multi-arm polyoxazolines. As shown schematically in FIG. 3, a reactive multi-arm polyoxazoline 210 as described above is crosslinked with a multifunctional compound 220 comprising functional groups that are reactive with the reactive groups of the multi-arm polyoxazoline 210 to form a crosslinked product 230.

In some embodiments, the reactive groups of the reactive multi-arm polyoxazoline are nucleophilic groups and the functional groups of the multifunctional compound group are electrophilic groups. In some embodiments, the reactive groups of the reactive multi-arm polyoxazoline are electrophilic groups and the functional groups of the multifunctional compound are nucleophilic groups.

For example, the functional groups of the multifunctional compound may be nucleophilic groups selected from amine groups and/or thiol groups. As another example, the functional groups of the multifunctional compound may be electrophilic groups selected from imidazole esters, imidazole carboxylates, benzotriazole esters, imide esters, including N-hydroxysuccinimidyl esters.

In various aspects, the present disclosure pertains to crosslinkable systems comprising reactive multi-arm polyoxazolines like those described herein and multifunctional compounds like those described herein.

In various aspects, the present disclosure pertains to crosslinked products of reactive multi-arm polyoxazolines like those described herein and multifunctional compounds like those described herein. Such crosslinked products may be formed in vivo or such crosslinked products may be formed ex vivo and subsequently administered to a subject. Such crosslinked products can be used in a wide variety of biomedical applications, including implants, lubricious coatings, and pharmaceutical compositions.

In various embodiments, the reaction of the functional groups of the multifunctional compound with the reactive end groups of the reactive multi-arm polyoxazoline results in amide linkages.

In certain beneficial embodiments, the multifunctional compounds for use in the present disclosure may be a polyamine compound. In general, polyamine compounds suitable for use in the present disclosure include, for example, small molecule polyamines (e.g., containing at least two amine groups, for instance, from 3 to 20 amine groups or more in certain embodiments), polymers having amine side groups, and branched polymers having amine end groups, including dendritic polymers having amine end groups. Polyamine compounds suitable for use in the present disclosure include those that comprises a plurality of —(CH₂)ₓ—NH₂ groups where x is 0, 1, 2, 3, 4, 5 or 6. Polyamine compounds suitable for use in the present disclosure include polyamine compounds that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Particular examples of polyamine compounds which may be used as the multifunctional compound include ethylenetriamine, diethylene triamine, hexamethylenetriamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, chitosan and derivatives thereof, and poly(allyl amine), among others among others.

In certain beneficial embodiments, the multifunctional compounds comprise one or more covalently linked radiopaque moieties, for example, bromine or iodine groups. For instance, a polyamine compound such as those described above, among others, may be linked to an iodine-containing moiety by a suitable covalent linkage, such as an ester or amide linkage, among others. Examples of such iodine-containing moieties include aromatic moieties that comprise a monocyclic or multicyclic aromatic structure, such as a benzene group or a naphthalene group, that is substituted with the following: one or more radiopaque functional groups, for example, one or more iodine groups and, optionally, a plurality of hydrophilic functional groups, for example, hydrophilic functional groups selected from one or more of hydroxyl groups, C₁-C₄-hydroxyalkyl groups, C₁-C₄-aminoalkyl groups or C₁-C₄-carboxyalkyl groups.

In embodiments where the reactive multi-arm polyoxazolines and/or the multifunctional compounds comprise one or more covalently linked radiopaque moieties, the crosslinked products of such reactive multi-arm polyoxazolines and/or multifunctional compounds are visible under fluoroscopy. In various embodiments, such crosslinked products have a radiopacity that is greater than 250 Hounsfield units (HU), beneficially anywhere ranging from 250 HU to 500 HU to 750 HU to 1000 HU or more (in other words, ranging between any two of the preceding numerical values).

As previously noted, in various aspects, the present disclosure pertains to crosslinkable systems that comprise a reactive multi-arm polyoxazoline like that described herein and multifunctional compound like that described herein. In certain embodiments, systems are provided that are configured to deliver a polyamine compound and a reactive multi-arm polyoxazoline that comprises a plurality of reactive end groups that are reactive with the amino groups of the polyamine compound, under conditions such that the polyamine compound and the reactive multi-arm polyoxazoline crosslink with one another. Such conditions include basic conditions, such as those having a pH ranging from about 9 to about 11, typically ranging from about 9.5 to about 10.5, and more typically ranging from about 9.8 to about 10.2.

In some embodiments, systems may be provided that comprise a first composition comprising a polyamine compound like that described herein and a second composition comprising a reactive multi-arm polyoxazoline like that described herein.

The first composition may be a first fluid composition comprising the polyamine compound or a first dry composition that comprises the polyamine compound, to which a suitable fluid such as water for injection, saline, etc. can be added to form a first fluid composition. In addition to the polyamine compound, the first composition may further comprise additional agents, including those described below.

The second composition may be a second fluid composition comprising the reactive multi-arm polyoxazoline or a second dry composition that comprises the reactive multi-arm polyoxazoline, to which a suitable fluid such as water for injection, saline, etc. can be added to form a second fluid composition. In addition to the reactive multi-arm polyoxazoline, the second composition may further comprise additional agents including as those described below.

In some embodiments, the polyamine compound is initially combined with the reactive multi-arm polyoxazoline at an acidic pH at which crosslinking between the reactive groups of the reactive multi-arm polyoxazoline and the amino groups of the polyamine compound is suppressed (e.g., a pH ranging, for example, from about 3 to about 5, typically ranging from about 3.5 to about 4.5, and more typically ranging from about 3.8 to about 4.2). Then, when crosslinking is desired, a pH of the mixture of the polyamine compound and the reactive multi-arm polyoxazoline is changed from an acidic pH to a basic pH (e.g., a pH ranging from about 9 to about 11, typically ranging from about 9.5 to about 10.5, and more typically ranging from about 9.8 to about 10.2), leading to crosslinking between the polyamine compound and the reactive multi-arm polyoxazoline.

In particular embodiments, the system comprises (a) a first precursor composition that comprises a polyamine compound as described herein, (b) a second precursor composition that comprises a reactive multi-arm polyoxazoline as described herein, and (c) a third composition, specifically, an accelerant composition, that contains an accelerant that is configured to accelerate crosslinking reaction between the polyamine compound and the reactive multi-arm polyoxazoline.

The first precursor composition may be a first fluid composition comprising the polyamine compound that is buffered to an acidic pH or a first dry composition that comprises the polyamine compound and acidic buffering composition, to which a suitable fluid such as water for injection, saline, etc. can be added to form a first fluid composition comprising the polyamine compound that is buffered to an acidic pH. In some embodiments, for example, the acidic buffering composition may comprise monobasic sodium phosphate, among other possibilities. The first fluid composition comprising the polyamine compound may have a pH ranging, for example, from about 3 to about 5, typically ranging from about 3.5 to about 4.5, and more typically ranging from about 3.8 to about 4.2. In addition to the polyamine compound, the first precursor composition may further comprise additional agents, such as therapeutic agents and/or imaging agents.

The second precursor composition may be a second fluid composition comprising the reactive multi-arm polyoxazoline or a second dry composition that comprises the reactive multi-arm polyoxazoline from which a fluid composition is formed, for example, by the addition of a suitable fluid such as water for injection, saline, or the first fluid composition comprising the polyamine compound that is buffered to an acidic pH. In addition to the reactive multi-arm polyoxazoline, the second precursor composition may further comprise additional agents, such as therapeutic agents and/or imaging agents.

In one embodiment, the first precursor composition is a first fluid composition comprising the polyamine compound that is buffered to an acidic pH and the second precursor composition comprises a dry composition that comprises the reactive multi-arm polyoxazoline. The first precursor composition may then be mixed with the second precursor composition to provide a prepared fluid composition that is buffered to an acidic pH and comprises the polyamine compound and the reactive multi-arm polyoxazoline. In a particular example, a syringe may be provided that contains a first fluid composition comprising the polyamine compound that is buffered to an acidic pH, and a vial may be provided that comprises a dry composition (e.g., a powder) that comprises the reactive multi-arm polyoxazoline. The syringe may then be used to inject the first fluid composition into the vial containing the reactive multi-arm polyoxazoline to form a prepared fluid composition that contains the polyamine compound and the reactive multi-arm polyoxazoline, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11, typically ranging from about 9.5 to about 10.5, and more typically ranging from about 9.8 to about 10.2. In addition to the above, the fluid accelerant composition may further comprise additional agents, such as therapeutic agents and/or imaging agents.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents for use in connection with x-ray fluoroscopy, including metals and metal compounds (e.g., metal salts, metal oxides, etc.), for instance, barium compounds, bismuth compounds and tungsten, among others, and iodinated compounds, among others, and radiocontrast agents based on the clinically important isotope ^{99m}Tc, as well as other gamma emitters such as ¹²³I, ¹²⁵I, ¹³¹I, ¹¹¹In, ⁵⁷Co, ¹⁵³Sm, ¹³³Xe, ⁵¹Cr, ^{81m}Kr, ²⁰¹Tl, ⁶⁷Ga, and ⁷⁵Se, among others, (e) positron emitters, such as ¹⁸F, ¹¹C, ¹³N, ¹⁵O, and ⁶⁸Ga, among others, may be employed to yield functionalized radiotracer coatings, and (f) contrast agents for use in connection with near-infrared (NIR) imaging, which can be selected to impart near-infrared fluorescence to the coatings of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others. NIR-sensitive dyes include cyanine dyes, squaraines, phthalocyanines, porphyrin derivatives and borondipyrromethane (BODIPY) analogs, among others.

A prepared fluid composition that is buffered to an acidic pH and comprises the polyamine compound and the reactive multi-arm polyoxazoline, as described above, and a fluid accelerant composition that is buffered to basic pH, as described above, may be combined form a crosslinked hydrogel, either in vivo or ex vivo.

In various embodiments, a system is provided that include one or more delivery devices for delivering first and second compositions to a subject.

In some embodiments, the system may include a delivery device that comprises a first reservoir that contains a first composition that comprises a polyamine compound as described above and a second reservoir that contains a second composition that comprises a reactive multi-arm polyoxazoline as described above. In some embodiments, the system may include a delivery device that comprises a first reservoir that contains a first composition that comprises the polyamine compound and the reactive multi-arm polyoxazoline and is buffered to an acidic pH, such as the prepared fluid composition previously described, and a second reservoir that contains a second composition, such as the fluid accelerant composition described above. In either case, during operation, the first composition and second composition are dispensed from the first and second reservoirs and combined, whereupon the polyamine compound and the reactive multi-arm polyoxazoline and crosslink with one another to form a hydrogel.

Regardless of the particular first and second compositions selected, in particular embodiments, the system may include a delivery device that comprises a double-barrel syringe, which includes first barrel having a first barrel outlet, which first barrel contains the first composition, a first plunger that is movable in the first barrel, a second barrel having a second barrel outlet, which second barrel contains the second composition, and a second plunger that is movable in the second barrel.

In some embodiments, the device may further comprise a mixing section having a first mixing section inlet in fluid communication with the first barrel outlet, a second mixing section inlet in fluid communication with the second barrel outlet, and a mixing section outlet. In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked hydrogel is ultimately formed at the administration location.

As seen from the above, the compositions of the present disclosure may be used in a variety of medical procedures, including the following, among others: a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue, or a procedure to implant a fiducial marker comprising a crosslinked product of the first and second fluid compositions.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

What is claimed is:

1. A reactive multi-arm polymer having a cage-like silicon-oxygen core and a plurality of polyoxazoline-containing arms extending from the core, wherein the polyoxazoline-containing arms comprise a first end that is covalently attached to the cage-like silicon-oxygen core and a second end comprising a moiety that comprises a reactive end group.

2. The reactive multi-arm polymer of claim 1, wherein the cage-like silicon-oxygen core is selected from a T₆ cage-like silicon-oxygen core, a T₈ cage-like silicon-oxygen core, a T₁₀ cage-like silicon-oxygen core and a T₁₂ cage-like silicon-oxygen core.

3. The reactive multi-arm polymer of claim 1, wherein the polyoxazoline-containing arms comprise polymerized monomers selected from oxazoline, 2-(C₁-C₁₀-alkyl)-2-oxazolines, and combinations thereof.

4. The reactive multi-arm polymer of claim 1, wherein the reactive end groups are electrophilic groups.

5. The reactive multi-arm polymer of claim 4, wherein the electrophilic groups are selected from N-hydroxysuccinimidyl esters, imidazole esters, imidazole carboxylates and benzotriazole esters.

6. The reactive multi-arm polymer of claim 1, wherein the moiety that comprises the reactive end group may further comprise a hydrolysable ester group.

7. The reactive multi-arm polymer of claim 1, wherein the moiety that comprises the reactive end group comprises a diester selected from a malonic-acid-based diester, a succinic-acid-based diester, a glutaric-acid-based diester and an adipic-acid-based diester.

8. A system that comprises (a) a reactive multi-arm polymer having a cage-like silicon-oxygen core and a plurality of polyoxazoline-containing arms extending from the core, wherein the polyoxazoline-containing arms comprise a first end that is covalently attached to the cage-like silicon-oxygen core and a second end comprising a moiety that comprises a reactive end group and (b) a multifunctional compound that comprises functional groups that are reactive with the reactive end groups of the reactive multi-arm polymer.

9. The system of claim 8, wherein the reactive groups of the reactive multi-arm polymer are electrophilic groups and the functional groups of the multifunctional compound are nucleophilic groups.

10. The system of claim 9, wherein the multifunctional compound is a polyamine compound.

11. The system of claim 10, wherein the polyamine compound comprises residues of from 2 to 10 basic amino acids.

12. The system of claim 10, wherein the polyamine compound comprises a plurality of —(CH₂)ₓ—NH₂ groups where x is 0, 1, 2, 3, 4, 5 or 6.

13. The system of claim 10, wherein the polyamine compound comprises two or more amino acid residues selected from residues of lysine, ornithine, and combinations thereof.

14. The system of claim 10, wherein the system comprises a first precursor composition that comprises the multifunctional compound and a second precursor composition that comprises the reactive multi-arm polymer.

15. The system of claim 14, further comprising an accelerant composition.

16. The system of claim 15, wherein the accelerant composition comprises a buffer solution having a pH ranging from about 9 to about 11.

17. The system of claim 16, wherein the first precursor composition is provided in a syringe barrel, the second precursor composition is provided in a vial, and the accelerant composition is provided in a syringe barrel.

18. The system of claim 8, further comprising a delivery device.

19. A medical hydrogel formed by crosslinking a reactive multi-arm polymer having a cage-like silicon-oxygen core and a plurality of polyoxazoline-containing arms extending from the core, wherein the polyoxazoline-containing arms comprise a first end that is covalently attached to the cage-like silicon-oxygen core and a second end comprising a moiety that comprises a reactive end group with a multifunctional compound that comprises functional groups that are reactive with the reactive end groups of the reactive multi-arm polymer.

20. A method of treatment comprising administering to a subject a mixture that comprises a reactive multi-arm polymer in accordance with claim 1 and a multifunctional compound that comprises functional groups that are reactive with the reactive end groups of the reactive multi-arm polymer.
